(19) United States
(12) Patent Application Publication
(10) Pub. No.: US 2026/0365631 A1
(43) Pub. Date: Nov. 19, 2026

(54) PROCESS FOR THE SELECTIVE CLEAVAGE OF C-SCF3 BONDS AND ANALOGUES

(71) Applicants: CENTRE NATIONAL DE LA RECHERCHE SCIENTIFIQUE, Paris (FR); INSTITUT NATIONAL DES SCIENCES APPLIQUEES DE ROUEN NORMANDIE, Saint-Etienne-du-Rouvray (FR); UNIVERSITÉ DE ROUEN-NORMANDIE, Mont Saint-Aignan (FR)

(72) Inventors: Tatiana BESSET, Mont Saint Aignan Cedex (FR); Thomas CASTANHEIRO, Malaunay (FR); Nobile ENZO, Massy (FR); Mathieu ARRIBAT, Paulhan (FR)

(21) Appl. No.: 19/487,246

(22) PCT Filed: May 30, 2024
(86) PCT No.: PCT/EP2024/064950

§ 371 Date: Nov. 24, 2025

(30) Foreign Application Priority Data
Jun. 1, 2023 (EP) 23305864.3

(51) Int. Cl. C07D 277/82; C07D 277/74; C07F 9/6539

(52) U.S. Cl. CPC C07D 277/82; C07D 277/74; C07F 9/6539

(57) ABSTRACT

The application relates to a process for the preparation of a compound A-R, by reacting a compound A-X, in the presence of a metallic complex including nickel, palladium or rhodium (I), a ligand, a nucleophile carrying one group R, and a solvent,
R being a hydrocarbon group, optionally including at least one heteroatom and/or at least one atom other than C or H,
A being selected from the group consisting of: (C₆-C₁₀)aryl groups, heteroaryl groups, and a vinyl compound, and
X being selected from the group consisting of: YCF₃, Y(O)ₙCF₃, YCF₂SO₂Ph, Y(O)ₙCF₂SO₂Ph, Y(O)ₙCF₂COOR, Y(O)ₙCF₂CONR₂, Y(O)ₙCF₂CH₂OH, Y(O)ₙCN, Y(O)ₙCHF₂, Y(O)ₙCF₂H, Y(O)ₙCF₂CₙF₂ₙ₊₁, and Y(O)ₙCF₂COR, Y being S or Se, n being 1 or 2, and R being (C₁-C₆)alkyl.

The present invention concerns a process for the selective cleavage of C—SCF₃ bonds and analogues of such bonds.

In a society concerned about the environment, earth and aquatic systems pollution and circular economy, development of new technologies to meet such objectives is nowadays a real challenge and a hot topic across the whole chemical industry.

The field of organofluorine chemistry is unavoidable and fluorinated compounds are highly represented in many fields such as materials science, pharmaceutical and agrochemical industries. Indeed, the incorporation of a fluorine atom or a fluorinated unit can modify the physico-chemical properties of organic molecules in a significant way explaining the interest of the scientific community regarding this research area.

However, the prevalence of these compounds raises the question of their fate and degradation. Recently, there has been a strong awareness from the scientific community and society regarding chlorofluorocarbons (CFCs), hydrofluorocarbons (HFCs) and per- and poly-fluoroalkyl substances (PFAs). It is now important to go even further and to look at the fate of emerging fluorinated groups that have become essential, such as derivatives containing a SCF₃ unit (e.g. Toltrazuril and Fipronil).

In this context, the development of tools allowing to valorize these fluorinated derivatives, which, once used, are then considered as waste by converting them into compounds with high added value would have an undeniable ecological impact and allow for cost reduction. It is therefore essential to develop innovative tools to meet this challenge and to remove this synthetic lock.

There is currently no technology allowing to selectively cleave C(sp²)-SRf bonds on (hetero)aromatic derivatives or vinyl positions or C(sp³)-SRf bonds and any progress would have a major impact from a scientific and environmental point of view.

N. Barbero et al., Organic Letters, 2012, 14, 796-799 (Supporting Information, S1-S78) relates to catalytic reductive cleavage of unactivated C—SMe bonds. As shown on page 797, left column, last paragraph, the SMe group, namely an electron-donating group on the sulfur atom, was reported as the most efficient in the reductive cleavage of C—S bond. The optimization process reported in Tables 1 to 6 of the supporting information (p. S3 to S5) was carried out for this specific thioether only. Table 7 (p. S5) of this document confirms that SMe group raises the highest yield with respect to cleavage of the C—S bond, while electron-attracting groups, such as Ac and CONMe₂, were inefficient.

Surprisingly, it has now been found that it was possible to selectively cleave C—S bonds and analogues of such bonds on organic molecules in the presence of an electron-attracting fluorinated group, such as CF₃ or analogous fluorine-containing groups.

One aim of the present invention is thus to provide a tool for the de-fluorination of fluorinated compounds.

One aim of the present invention is to provide a process for the de-fluorination of fluorinated compounds, for example through the cleavage of C—SCF₃ bonds, or analogues of such bonds.

The present invention relates to a process for the preparation of a compound having the formula (I):

A-R
by reacting a compound having the formula (II):

A-X
in the presence of a metallic complex comprising nickel, palladium or rhodium(I), a ligand, a nucleophile carrying one group R, and a solvent, R being a hydrocarbon group, optionally including at least one heteroatom such as S, N or O, and/or at least one atom other than C or H, such as Se, Si, B, or P,
wherein:
   A is selected from the group consisting of:
      (C₆-C₁₀)aryl groups,
      said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
      Rₐ and R_{b} being independently from each other H or a (C₁-C₆)alkyl group;
      R_{c} being a (C₁-C₆)alkyl group;
      heteroaryl groups comprising from 5 to 10 atoms and including at least one heteroatom selected from O, N, and S,
      said heteroaryl groups being optionally substituted with at least one substituent preferably selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
      a vinyl compound having the following formula (III):

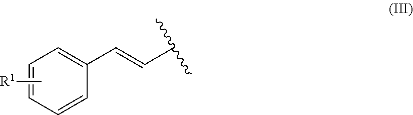

R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R, being as defined above; and
X is selected from the group consisting of: YCF₃, YOCF₃, YO₂CF₃, YCF₂SO₂Ph, YOCF₂SO₂Ph, YO₂CF₂SO₂Ph, Y(O)ₙCF₂COOR′, Y(O)ₙCF₂CONR′₂, Y(O)ₙCF₂CH₂OH, Y(O)ₙCN, Y(O)ₙCHF₂, Y(O)ₙCF₂H, Y(O)ₙCF₂CₙF₂ₙ₊₁, Y(O)ₙCF₂COR′, Y being S or Se,
n being 1 or 2, and R′ being (C₁-C₆)alkyl.

Thus, the present invention relates to a process for the preparation of a compound having the formula (I):

A-R
by reacting a compound having the formula (II):

A-X
in the presence of a metallic complex comprising nickel, a ligand, a nucleophile carrying one group R, and a solvent,
R being a hydrocarbon group, optionally including at least one heteroatom such as S, N or O, and/or at least one atom other than C or H, such as Se, Si, B, or P,
wherein:
   A is selected from the group consisting of:
      (C₆-C₁₀)aryl groups,
      said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
      Rₐ and R_{b} being independently from each other H or a (C₁-C₆)alkyl group; R_{c} being a (C₁-C₆)alkyl group;
      heteroaryl groups comprising from 5 to 10 atoms and including at least one heteroatom selected from O, N, and S,
      said heteroaryl groups being optionally substituted with at least one substituent preferably selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R, being as defined above; and
      a vinyl compound having the following formula (III):

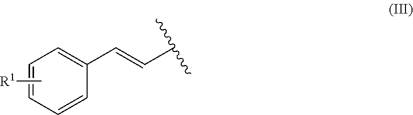

R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R, being as defined above; and
X is selected from the group consisting of: YCF₃, YOCF₃, YO₂CF₃, YCF₂SO₂Ph, YOCF₂SO₂Ph, YO₂CF₂SO₂Ph, Y(O)ₙCF₂COOR′, Y(O)ₙCF₂CONR′₂, Y(O)ₙCF₂CH₂OH, Y(O)ₙCN, Y(O)ₙCHF₂, Y(O)ₙCF₂H, Y(O)ₙCF₂CₙF₂ₙ₊₁, Y(O)ₙCF₂COR′, Y being S or Se,
n being 1 or 2, and R′ being (C₁-C₆)alkyl.

According to an embodiment, in formula (II) above, X is selected from the group consisting of: SCF₃, SOCF₃, SO₂CF₃, SCF₂SO₂Ph, SOCF₂SO₂Ph, SO₂CF₂SO₂Ph, S(O)ₙCF₂COOR, S(O)ₙCF₂CONR₂, S(O)ₙCF₂CH₂OH, S(O)ₙCN, S(O)ₙCHF₂, S(O)ₙCF₂H, S(O)ₙCF₂CₙF₂ₙ₊₁, S(O)ₙCF₂COR, and all analogues of this list in the selenium series, R and n being as defined above.

According to an embodiment, in formula (II) above, X is selected from the group consisting of: SCF₃, SOCF₃, SO₂CF₃, SCF₂SO₂Ph, SOCF₂SO₂Ph, SO₂CF₂SO₂Ph, S(O)ₙCF₂COOR, S(O)ₙCF₂CONR₂, S(O)ₙCF₂CH₂OH, S(O)ₙCN, S(O)ₙCHF₂, S(O)ₙCF₂H, S(O)ₙCF₂CₙF₂ₙ₊₁, S(O)ₙCF₂COR, and SeCF₃.

According to an embodiment, in formula (II) above, X is selected from the group consisting of: SCF₃, SOCF₃, SO₂CF₃, SCF₂SO₂Ph, SO₂CF₂SO₂Ph, and SeCF₃.

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms.

The term “alkyl group” means: a linear or branched, saturated, hydrocarbon-based aliphatic group comprising, unless otherwise mentioned, from 1 to 12 carbon atoms. By way of examples, mention may be made of methyl, ethyl, n-propyl, isopropyl, butyl, isobutyl, tert-butyl or pentyl groups.

The term “aryl group” means: a cyclic aromatic group comprising between 6 and 10 carbon atoms. By way of examples of aryl groups, mention may be made of phenyl or naphthyl groups.

The term “heteroaryl group” means: a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl.

Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

When an alkyl group is substituted with an aryl group, the term “arylalkyl” or “aralkyl” group is used. The “arylalkyl” or “aralkyl” groups are aryl-alkyl- groups, the aryl and alkyl groups being as defined above. Among the arylalkyl groups, mention may in particular be made of the benzyl or phenethyl groups.

The term “halogen” means: a fluorine, a chlorine, a bromine or an iodine.

The term “alkoxy group” means: an —O-alkyl group where the alkyl group is as previously defined. By way of examples, mention may be made of —O—(C₁-C₄)alkyl groups, and in particular the —O-methyl group, the —O-ethyl group, as —O—C₃alkyl group, the —O-propyl group, the —O-isopropyl group, and as —O—C₄alkyl group, the —O-butyl, —O-isobutyl or —O-tert-butyl group.

The term “alkynyl” as employed herein includes unsaturated, nonaromatic, hydrocarbon groups having 2 to 6 carbons, and comprising at least one triple bond.

Preferably, the alkynyl group is linear. Preferably, the alkynyl group is a —(CH₂)ₘ—C≡CH group, m being an integer comprised from 1 to 4.

The term “alkenyl” as employed herein includes unsaturated, nonaromatic, hydrocarbon groups having 2 to 6 carbons, and comprising at least one double bond. Preferably, the alkenyl group is linear. Preferably, the alkenyl group is a —(CH₂)ₘ—CH═CH₂ group, m being an integer comprised from 1 to 4.

The abovementioned “alkyl”, “aryl”, and “heteroaryl” groups can be substituted with one or more substituents. Among these substituents, mention may be made of the following groups: amino, amide, hydroxyl, thiol, oxo, halogen, alkyl, alkoxy, alkylthio, alkylamino, aryloxy, arylalkoxy, cyano, trifluoromethyl, carboxy or carboxyalkyl.

The term “alkylthio” means: an —S-alkyl group, the alkyl group being as defined above.

The term “alkylamino” means: an —NH-alkyl group, the alkyl group being as defined above.

The term “aryloxy” means: an —O-aryl group, the aryl group being as defined above.

The term “arylalkoxy” means: an aryl-alkoxy- group, the aryl and alkoxy groups being as defined above.

The term “carboxyalkyl” means: an HOOC-alkyl- group, the alkyl group being as defined above. As examples of carboxyalkyl groups, mention may in particular be made of carboxymethyl or carboxyethyl.

The term “haloalkyl group” means: an alkyl group as defined above, in which one or more of the hydrogen atoms is(are) replaced with a halogen atom. By way of example, mention may be made of fluoroalkyls, in particular CF₃ or CHF₂.

The term “carboxyl” means: a COOH group.

The term “oxo” means: “═O”.

The term “hydrocarbon group” means a group comprising hydrogen and carbon atoms. As mentioned above, R is a group comprising at least one carbon atom and at least one hydrogen atom. This group may also include at least one heteroatom, such as O, N or S, and/or at least one atom other than C or H, such as Se, Si, B or P.

In one embodiment of the process according to the invention, the nucleophile is selected from the group consisting of: alkyl amines, cycloalkyl amines, aryl amines, alkyl thiols, thiophenol compounds, aryl selenol compounds, phosphine oxides, Grignard reagents, alcohols, esters and phosphorus derivatives. The alkyl amines, cycloalkyl amines and aryl amines can be either primary or secondary amines. The terms “alkyl”, “cycloalkyl”, and “aryl” are as defined above.

Preferably, the nucleophile is a RH group, wherein R is selected from the group consisting of: (C₁-C₆)alkyl, heterocycloalkyl, —S-cyclo(C₃-C₁₀)alkyl, —O—(C₁-C₁₂)alkyl, an optionally substituted heteroaryl, —NR¹R², —P(═O)—R¹R², —X¹—Ar¹, —X¹—(C₁-C₁₂)alkyl-Ar¹, —X¹-Het¹, —X¹—(C₁-C₁₂)alkyl-Het¹, X¹ being S, Se, NH or N-alkyl, Ar¹ being an optionally substituted (C₆-C₁₀)aryl group, Het¹ being an optionally substituted heteroaryl group and R¹ and R² being, independently from each other, a (C₁-C₆)alkyl group or a (C₆-C₁₀)aryl group.

In particular, when the nucleophile is a cycloalkyl amine, R can be a
cyclo(C₃-C₁₀)alkylamino group as defined above.

In particular, when the nucleophile is a thiol, R can be —S—(C₁-C₆)alkyl (“alkylthio” as defined above), the alkyl being optionally substituted as defined above.

In particular, R can be (C₁-C₆)alkyl, S-cyclo(C₃-C₁₀)alkyl, O—(C₁-C₁₂)alkyl (“alkoxy” as defined above), —X¹—(C₁-C₁₂)alkyl-Ar¹, X¹—(C₁-C₁₂)alkyl-Het¹, where —X¹, Ar¹ and Het¹ are as defined above, and where the alkyl is optionally substituted by at least one substituent as defined above.

The terms “alkyl”, “aryl”, “heterocycloalkyl”, heteroaryl” and their optional substituents are as defined above.

Preferably, R is selected from the group consisting of: (C₁-C₆)alkyl, heterocycloalkyl, —S-cyclo(C₃-C₁₀)alkyl, —P(═O)—R¹R², and —X¹—Ar¹, X¹ being S, Se or NH, and Ar¹ being an optionally substituted (C₆-C₁₀)aryl group, and R¹ and R² being, independently from each other, a (C₁-C₆)alkyl group or a (C₆-C₁₀)aryl group.

More preferably, the nucleophile is selected from the group consisting of: heterocycloalkyl groups comprising at least one nitrogen and/or sulfur atom such as morpholine, aniline groups and derivatives thereof, such as 4-methylaniline,

H—S-(cyclo)alkyl groups, such as CySH, H—S-aryl groups, such as 4-methylthiophenol,

H—Se-aryl groups, such as benzene selenol, phosphine oxides such as HP(═O)Ph₂, Grignard reagents such as Alk-MgBr, in particular iPrMgBr and phosphorus derivatives such as HPPh₂ where Ph is phenyl.

In one embodiment, in formula (I) and in formula (II) as defined above, A is selected from the heteroaryl groups comprising from 5 to 10 atoms and including at least one heteroatom selected from O, N, and S, said heteroaryl groups being optionally substituted with at least one substituent selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R, being as defined above.

According to one embodiment, the process of the invention as defined above further comprises the use of a hydride compound, such as LiHMDS. In this embodiment, the compound having the formula (II) is reacted with the nucleophile in the presence of the metallic complex comprising nickel, palladium or rhodium (1) the ligand, and the solvent, and also with a hydride compound.

In this embodiment, the nucleophile is in particular a Grignard reactant.

According to one embodiment, in the process according to the invention, the ligand is selected from the group consisting of: 1,5-cyclooctadiene (COD), 1,2-Bis(dicyclohexylphosphino)ethane (dcype), 1,1′-Bis(diphenylphosphino)ferrocene (dppf), 1,1′-Bis(di-tert-butylphosphino)ferrocene (DTBPF), 4,5-Bis(diphenylphosphino)-9,9-dimethylxanthene (XantPhos), triphenylarsine (AsPh₃) and triphenylphosphane (PPh₃).

The term “ligand” refers to a chemical component that is able to bind to the metal of the metallic complex. The initial state of a metallic complex is the state where the metallic complex is used in the reaction medium. In the initial state of the metallic complex, the ligand can be bound to the metal.

Alternatively, the ligand can be added to the reaction medium in addition to the metallic complex. In such case, the ligand will bind to the metal in the reaction medium.

According to an embodiment of the process, the ligand is bound to the metal in the initial state of the metallic complex.

According to another embodiment of the process, the ligand is added to the reaction medium in addition to the metallic complex.

According to a further embodiment of the process, at least one first ligand is bound to the metal in the initial state of the metallic complex and at least one second ligand is added to the reaction medium in addition to the metallic complex, wherein the first and second ligands are identical or different.

Preferably, the metallic complex is a Ni(0) complex such as bis(1,5-cyclooctadiene)nickel (Ni[cod]₂) or bis(triphenylphosphine)nickel (Ni[PPh₃]2).

According to another embodiment, the metallic complex comprises nickel, palladium or rhodium(I). For example, the metallic complex can be a Pd(0) complex such as tris(dibenzylideneacetone)dipalladium(0) of formula Pd₂dba₃ or tetrakis(triphenylphosphine)palladium(0) of formula Pd(PPh₃)₄. In particular, the metallic complex can be a Pd(II) complex such as, for example, [1,3-Bis(2,6-Diisopropylphenyl)imidazol-2-ylidene](3-chloropyridyl)palladium(II) dichloride (PEPPSI™-SIPr catalyst, 905459-27-0), palladium(II) acetate of formula Pd(OAc)₂ or allylpalladium(II) chloride dimer of formula [Pd(π-allyl)Cl]₂. Alternatively, the metallic complex can be a Rh(I) complex such as, for example, rhodium carbonyl chloride of formula [Rh(CO)₂Cl]₂ or a hydroxy(cyclooctadiene)rhodium(I) dimer of formula [Rh(OH)COD]₂.

According to one embodiment, the solvent used in the process according to the invention is selected from the usual solvents used in the field. Preferably, the solvent for the process according to the invention is toluene.

According to one embodiment of the process according to the invention, the reaction is carried out a temperature from 90 °C to 140 °C. Preferably, the reaction may be carried out at 90 °C, 110 °C, 120 °C, and 140 °C.

The present invention also relates to the process as defined above, for the preparation of a compound having the formula (I-1):

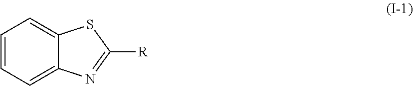

R being as defined above,
from a compound having the formula (II-1):

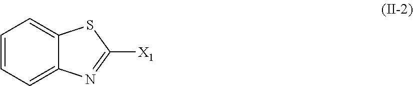

X₁ being selected from the group consisting of: SCF₃, SOCF₃, SO₂CF₃, SCF₂SO₂Ph, SOCF₂SO₂Ph, SO₂CF₂SO₂Ph, and SeCF₃.

### EXAMPLES

### Example 1: Selective Cleavage of C—SCF₃ Bond in the Presence of an Alkyl Amine as a Nucleophile

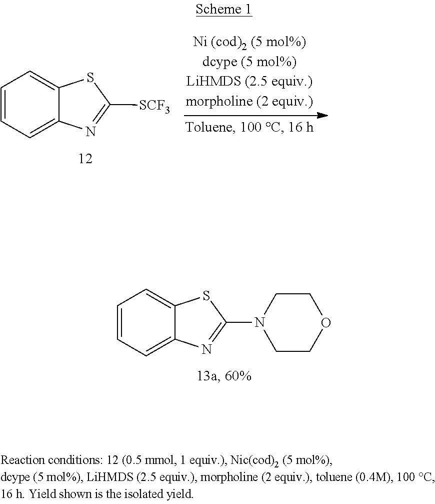

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), alkyl amine (1.25 mmol, 2 equiv.) and toluene (1.25 mL) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 13a.

The result shows that the cleavage of the C—S bond in the presence of a CF₃ group is achieved with a good yield when using an alkylamine as a nucleophile.

### Example 2: Selective Cleavage of C—SCF₃ Bond in the Presence of an Aryl Amine as a Nucleophile

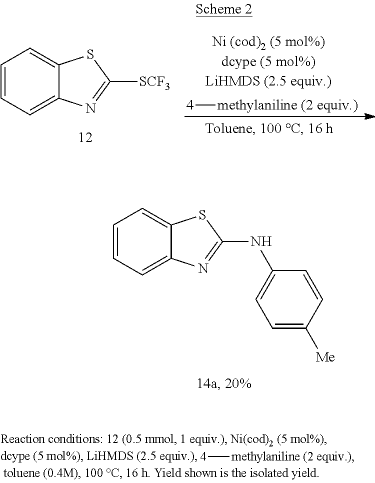

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), aryl amine (1.25 mmol, 2 equiv.) and toluene (1.25 mL) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired product 14a.

The result shows that primary and aromatic amines are suitable nucleophiles in this Ni-catalyzed transformation allowing the cleavage of the C—S bond in the presence of a CF₃ group.

### Example 3: Selective Cleavage of C—SCF₃ Bond in the Presence of an Alkyl Thiol as a Nucleophile

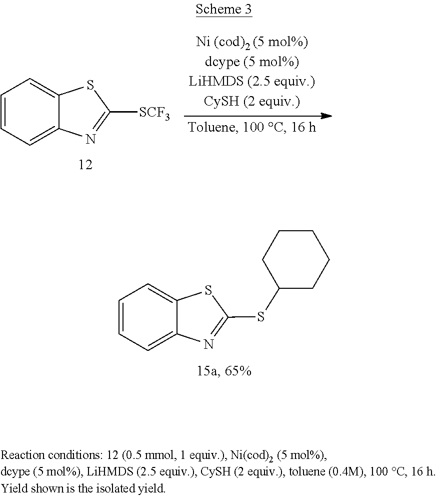

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), alkyl thiol (1.25 mmol, 2 equiv.) and toluene (1.25 mL) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired product 15a.

The result shows that the SCF₃ group was selectively replaced by a group consisting of a sulfur atom linked to an alkyl (SAlkyl group) with a high yield.

### Example 4: Selective Cleavage of C—SCF₃ Bond in the Presence of a Thiophenol Derivative as a Nucleophile

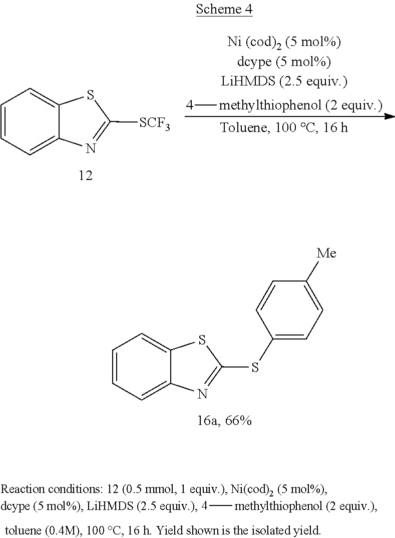

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), aryl thiol (1.25 mmol, 2 equiv.) and toluene (1.25 mL) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 16a.

The SCF₃ moiety was also successfully replaced by a group consisting of a sulfur atom linked to an aryl (SAr group). These results showed that chalcogens are well compatible as class of nucleophiles.

### Example 5: Selective Cleavage of C—SCF₃ Bond in the Presence of an Aryl Selenol as a Nucleophile

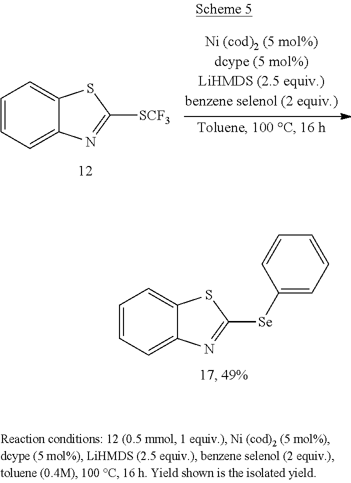

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), aryl selenol (1.25 mmol, 2 equiv.) and toluene (1.25 mL) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 17.

The results show that an aryl selenol is also a suitable nucleophile.

### Example 6: Selective Cleavage of C—SCF₃ Bond in the Presence of a Phosphine Oxide as Nucleophile

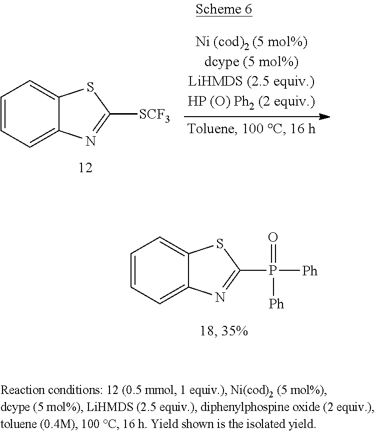

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.), 2-((trifluoromethyl)thio)benzo[d]thiazole 12 (118 mg, 0.5 mmol, 1 equiv.), phosphine oxide (1.25 mmol, 2 equiv.) and toluene (1.25 mL) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired product 18.

The result shows that phosphine oxides are suitable nucleophiles in this Ni-catalyzed transformation.

### Example 7: Selective Cleavage of C—SCF₃ Bond in the Presence of a Grignard Reagent as a Nucleophile

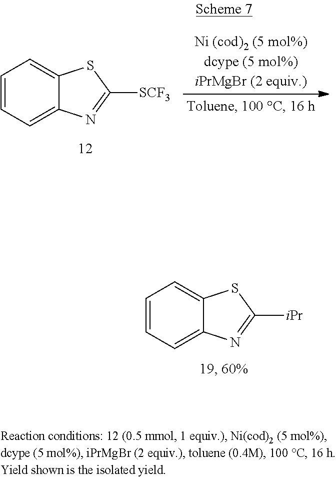

An oven-dried 10 mL tube equipped with a stirring bar was charged with Ni(cod)₂ (7 mg, 0.025 mmol, 5 mol %), dcype (11 mg, 0.025 mmol, 5 mol %), 2-((trifluoromethyl)thio)benzo[d]thiazole 12 (118 mg, 0.5 mmol, 1 equiv.), Grignard reagent (1.25 mmol, 2 equiv.) and toluene (1.25 mL) under argon. The resulting solution was stirred at 100 °C for 16 h. The mixture was allowed to cool down to 21 °C. The residue was concentrated under vacuum and purified by silica gel flash column chromatography to isolate the desired product 19.

The result shows that a Grignard reagent is a suitable nucleophile for the replacement of a SCF₃ group by an alkyl.

### Example 8: Selective Cleavage of C—SOCF₃ Bond

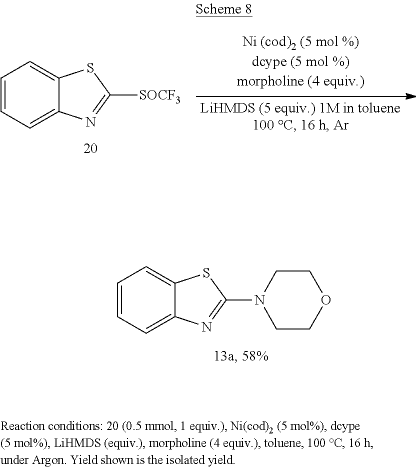

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS in toluene (2.5 mL, 1M, 418 mg, 2.5 mmol, 5 equiv.), 20 (0.5 mmol, 1 equiv.) and morpholine (2 mmol, 4 equiv.) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired product 13a.

The result shows that when using an aliphatic amine (morpholine) as a nucleophile, the cleavage of the C—S(O)SCF₃ bond, where the carbon atom is linked to a sulfoxide, is achieved with a good yield.

### Example 9: Selective Cleavage of C—SCF₂SO₂Ph Bond

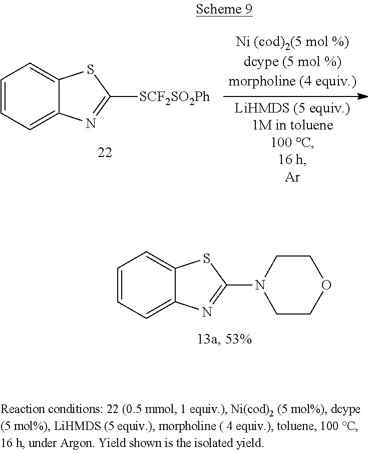

An oven-dried 10 mL tube equipped with a stirring bar was charged with Ni(cod)₂ (4 mg, 0.015 mmol, 5 mol %), dcype (6 mg, 0.015 mmol, 5 mol %), LiHMDS in toluene (1.5 mL, 1M, 251 mg, 1.5 mmol, 5 equiv.), 22 (107 mg, 0.3 mmol, 1 equiv.) and morpholine (1.2 mmol, 4 equiv.) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 13a. The product and the corresponding yield are reported in scheme 9.

The result shows that the process according to the invention allows the cleavage of a C—S bond bearing another fluorinated functional group, namely CF₂SO₂Ph group, using an amine (morpholine) as a nucleophile.

### Example 10: Selective Cleavage of C—SCF₃ Bond in the Presence of Various Metallic Complexes and Various Ligands

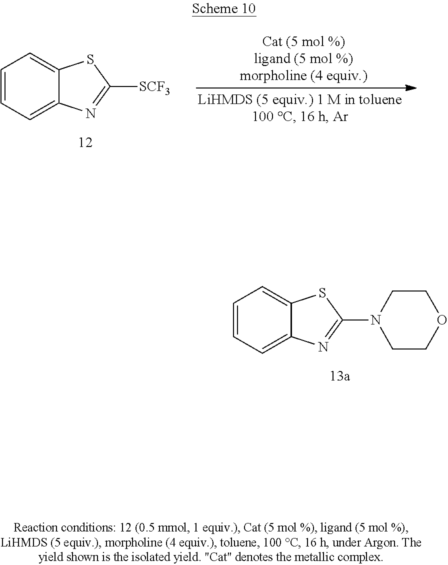

For 10-A and 10-C, an oven-dried 10 mL tube equipped with a stirring bar was charged with Ni(cod)₂ (7 mg, 0.025 mmol, 5 mol %) or Ni(PPh₃)₂ (14.6 mg, 0.025 mmol, 5 mol %), dcype (11 mg, 0.025 mmol, 5 mol %), LiHMDS in toluene (2.5 mL, 1M, 418 mg, 2.50 mmol, 5 equiv.), 2-((trifluoromethyl)thio)benzo[d]thiazole 12 (118 mg, 0.5 mmol, 1 equiv.), morpholine (2.5 mmol, 4 equiv.) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum.

In the case of 10-A, the residue was purified by silica gel flash column chromatography to isolate the desired product 13a. For 10-B, 10-C and 10-D, yields were determined by ¹H NMR with 1,3,5-Trimethoxybenzene as an internal standard. The product obtained and the corresponding yields are reported in scheme 10 and Table 1, respectively.

The same procedure was followed for 10-B and 10-D except that dcype was not added to the solution.

The metallic complexes, ligands and reaction yields are reported in Table 1.

TABLE 1

| Entry | Metallic complex (cat) | Ligand | Reaction yield |
|---|---|---|---|
| 10-A | Ni(cod)₂ | dcype | 71% (isolated yield) |
| 10-B | Ni(cod)₂ | — | 49% (¹H NMR) |
| 10-C | Ni(PPh₃)₂ | dcype | 47% (¹H NMR) |
| 10-D | Ni(PPh₃)₂ | — | 53% (¹H NMR) |

The results show that the reaction was smoothly conducted using the metallic complexes with or without the addition of dcype as a ligand.

### Example 11: Selective Cleavage of C—SCF₃ Bond in the Presence of a Pd Complex

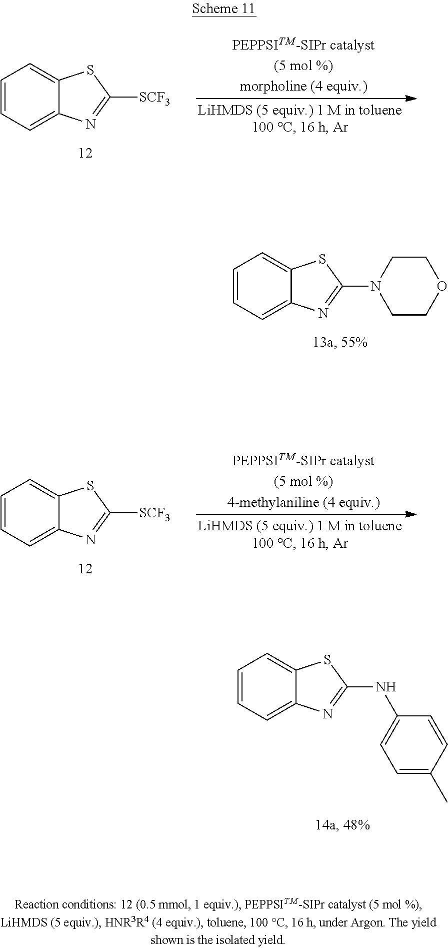

An oven-dried 10 mL tube equipped with a stirring bar was charged with [1,3-Bis(2,6-Diisopropylphenyl)imidazol-2-ylidene](3-chloropyridyl)palladium(II) dichloride (PEPPSI™-SIPr catalyst, 905459-27-0, 17 mg, 0.025 mmol, 5 mol %)) of formula:

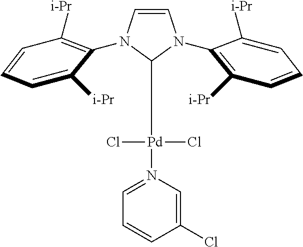

with LiHMDS 1M in Toluene (2.5 mL, 418 mg, 2.50 mmol, 5 equiv., 1M), 2-((trifluoromethyl)thio)benzo[d]thiazole 12 (118 mg, 0.5 mmol, 1 equiv.) and morpholine or 4-methylaniline (2 mmol, 4 equiv.) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 13a or 14a.

The result shows that the C—SCF₃ bond cleavage can be achieved when using a Pd-catalyst in the presence of a secondary aliphatic amine or an aniline derivative.

### Example 12: Selective Cleavage of C—SCF3 Bond in the Presence of a Ligand and a Pd Complex

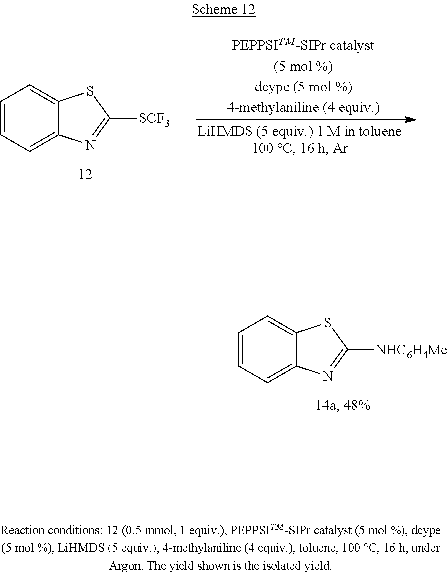

An oven-dried 10 mL tube equipped with a stirring bar was charged with [1,3-Bis(2,6-Diisopropylphenyl)imidazol-2-ylidene](3-chloropyridyl)palladium(II) dichloride (PEPPSI™-SIPr catalyst, 905459-27-0, 17 mg, 0.025 mmol, 5 mol %)), dcype (11 mg, 0.025 mmol, 5 mol %), LiHMDS 1M in toluene (2.5 mL, 418 mg, 2.50 mmol, 5 equiv., 1M), 2-((trifluoromethyl)thio)benzo[d]thiazole 12 (118 mg, 0.5 mmol, 1 equiv.) and 4-methylaniline (2 mmol, 4 equiv.) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic phase were dried over MgSO₄ and concentrated under vacuum. The residue was purified by silica gel flash column chromatography to isolate the desired product 14a.

The results show that the C—S bond cleavage of 12 is achieved whether the ligand is bound to the metal in the initial state of the metallic complex) (Example 11) or when the ligand is added to the reaction medium in addition to the metallic complex (Example 12).

### Example 13: Selective Cleavage of C—SCF₃ Bond in the Presence of a Primary Amine as a Nucleophile

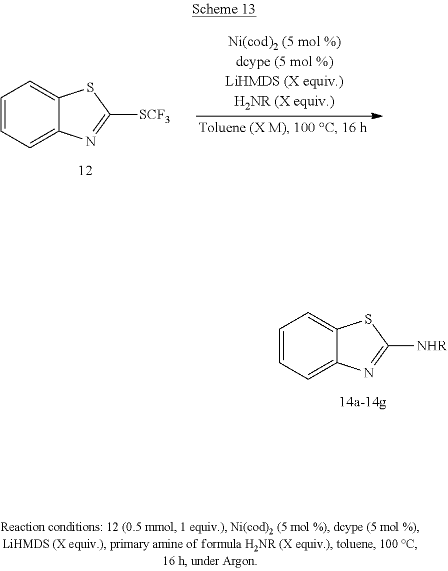

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.; or 418 mg, 2.5 mmol, 5 equiv.), 2-((trifluoromethyl)thio)benzo[d]thiazole 12 (118 mg, 0.5 mmol, 1 equiv.), primary amine of formula H₂NR (2 mmol, 4 equiv.) and toluene under argon. The reaction conditions are reported in Table 2 below. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired products. The products and the corresponding isolated yields are reported in scheme 14 and in Table 2 below.

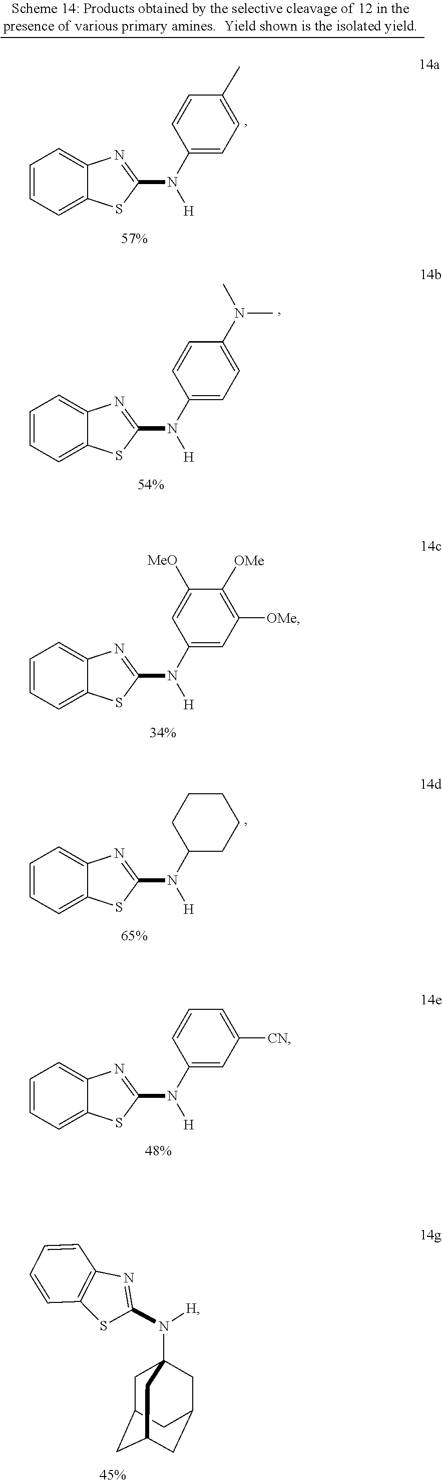

TABLE 2

| | H₂NR | LiHMDS | Toluene | Yield |
|---|---|---|---|---|
| Product | (X eq) | (X eq) | (X M) | (%) |
| 14-a | 4 | 5 | 0.4 | 57 |
| 14-b | 4 | 5 | 0.4 | 54 |
| 14-c | 2 | 2.5 | 0.4 | 34 |
| 14-d | 4 | 5 | 0.4 | 65 |
| 14-e | 4 | 5 | 0.4 | 48 |
| 14-g | 4 | 5 | 0.2 | 45 |

### Example 14: Selective Cleavage of C—SCF₃ Bond in the Presence of a Secondary Amine as a Nucleophile

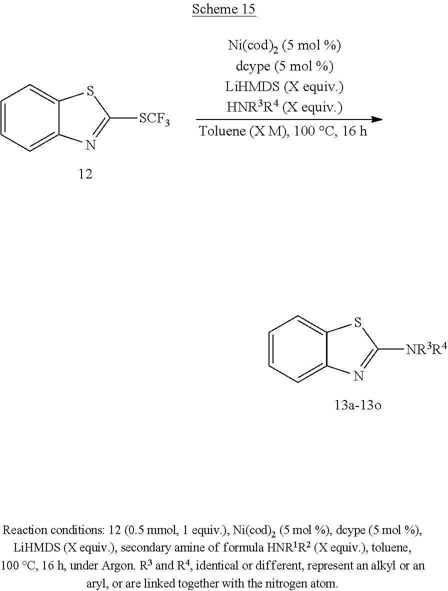

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.; or 418 mg, 2.5 mmol, 5 equiv.), 2-((trifluoromethyl)thio)benzo[d]thiazole 12 (118 mg, 0.5 mmol, 1 equiv.), secondary amine of formula HNR¹R² (2 mmol, 4 equiv.) and toluene under argon. The reaction conditions are reported in Table 3 below. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic phase were dried over MgSO₄ and concentrated under vacuum. The residue was purified by silica gel flash column chromatography to isolate the desired products. The products and the corresponding yields are reported in scheme 16 and in Table 3 below.

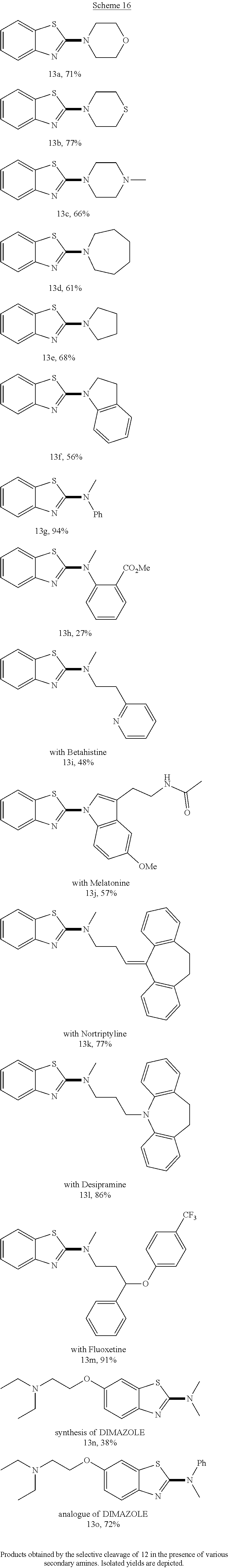

TABLE 3

| | 12 | HNR¹R² | LiHMDS | Toluene | Yield |
|---|---|---|---|---|---|
| Product | (X mmol) | (X eq) | (X eq) | (X M) | (%) |
| 13-a | 0.5 | 4 | 5 | 0.2 | 71 |
| 13-b | 0.5 | 2 | 2.5 | 0.4 | 77 |
| 13-c | 0.5 | 2 | 2.5 | 0.4 | 66 |
| 13-d | 0.5 | 2 | 2.5 | 0.4 | 61 |
| 13-e | 0.5 | 4 | 5 | 0.4 | 66 |
| 13-f | 0.5 | 2 | 2.5 | 0.4 | 56 |
| 13-g | 0.5 | 4 | 5 | 0.2 | 94 |
| 13-h | 0.5 | 4 | 5 | 0.4 | 27 |
| 13-i | 0.5 | 4 | 5 | 0.2 | 48 |
| 13-j | 0.5 | 4 | 5 | 0.4 | 57 |
| 13-k | 0.5 | 4 | 5 | 0.2 | 77 |
| 13-l | 0.5 | 4 | 5 | 0.2 | 86 |
| 13-m | 0.5 | 4 | 5 | 0.2 | 91 |
| 13-n | 0.5 | 4 | 5 | 0.2 | 38 |
| 13-o | 0.2 | 4 | 5 | 0.2 | 72 |

### Example 15: Selective Cleavage of C—SCF₃ Bond in the Presence of a Thiol as a Nucleophile

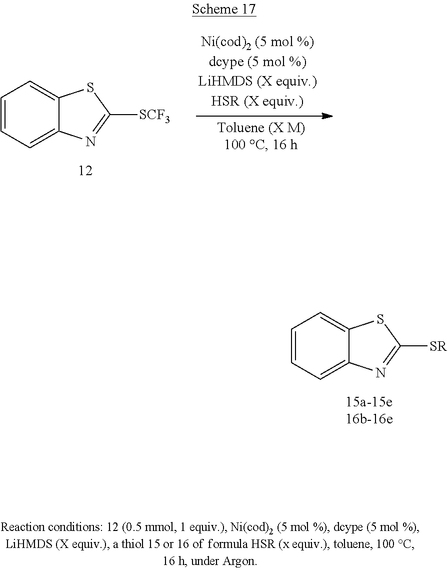

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (209 mg, 1.25 mmol, 2.5 equiv.; or 418 mg, 2.5 mmol, 5 equiv.), 2-((trifluoromethyl)thio)benzo[d]thiazole 12 (118 mg, 0.5 mmol, 1 equiv.), a thiol 15 or 16 of formula HSR (2 mmol, 4 equiv.), and toluene under argon. The reaction conditions are reported in Table 4 below. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic phase were dried over MgSO₄ and concentrated under vacuum. The residue was purified by silica gel flash column chromatography to afford the desired products. The products and the corresponding yields are reported in scheme 18 and in Table 4 below.

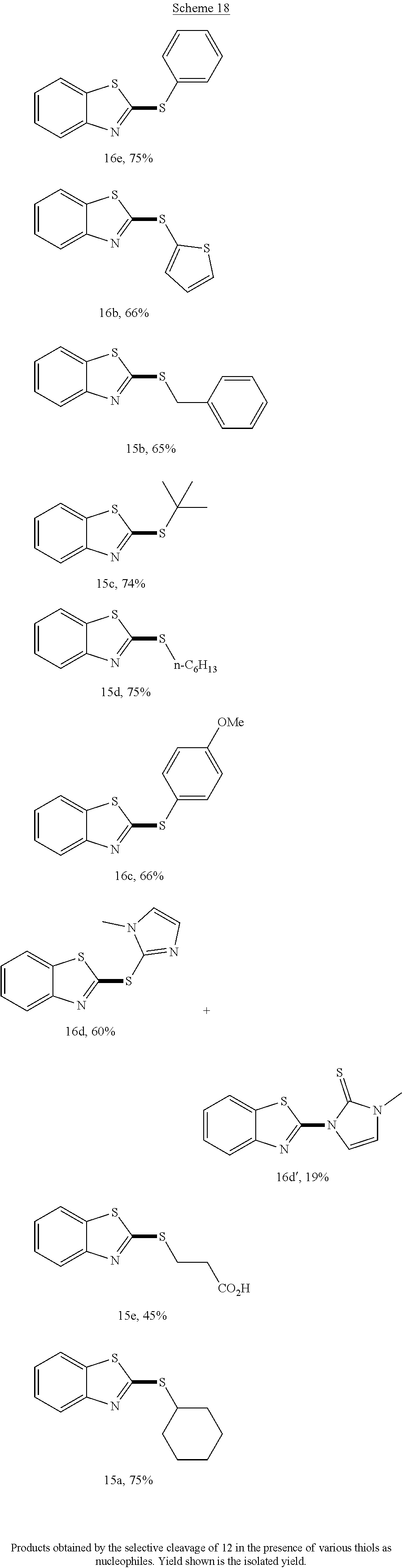

TABLE 4

| | 12 | Thiol | LiHMDS | Toluene | Yield |
|---|---|---|---|---|---|
| Product | (X mmol) | (X eq) | (X eq) | (X M) | (%) |
| 16e | 0.5 | 4 | 5 | 0.4 | 75 |
| 16b | 0.5 | 4 | 5 | 0.4 | 66 |
| 15b | 0.5 | 4 | 5 | 0.4 | 65 |
| 15c | 0.5 | 4 | 5 | 0.4 | 74 |
| 15d | 0.5 | 4 | 5 | 0.4 | 75 |
| 16c | 0.5 | 4 | 5 | 0.4 | 66 |
| 16d | 0.5 | 4 | 5 | 0.4 | 60 |
| 16d′ | | | | | 19 |
| 15e | 0.5 | 4 | 5 | 0.4 | 45 |
| 15a | 0.5 | 4 | 5 | 0.2 | 75 |

### Example 16: Selective Cleavage of C—SCF₃ Bond in the Presence of a Phosphorus Derivate as a Nucleophile

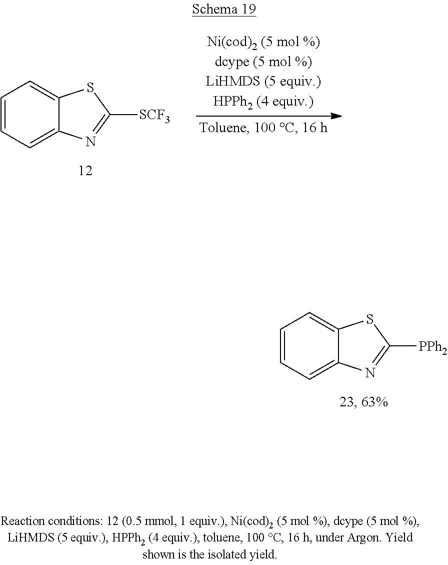

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS (418 mg, 2.5 mmol, 5 equiv.), 2-((trifluoromethyl)thio)benzo[d]thiazole 12 (118 mg, 0.5 mmol, 1 equiv.), HPPh₂ (2 mmol, 4 equiv.), and toluene under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic phase were dried over MgSO₄ and concentrated under vacuum. The residue was purified by silica gel flash column chromatography to isolate the desired product 23.

This result shows that the cleavage of 12 is achieved with a high yield in presence of a phosphorus nucleophile and shows an interesting swap between S- and P-containing groups.

### Example 17: Selective Cleavage of C—SCF₃ Bond in the Presence of an Alcohol as a Nucleophile

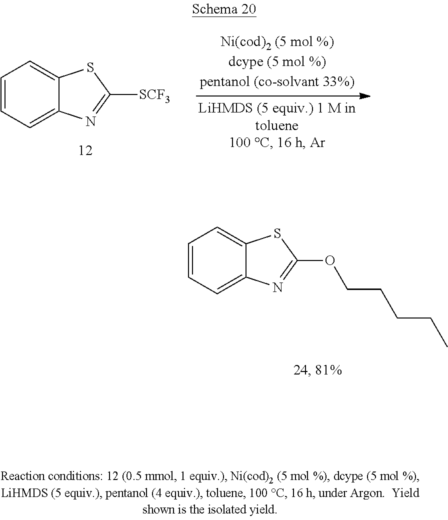

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS in toluene (2.5 mL, 1M, 418 mg, 2.5 mmol, 5 equiv.), 2-((trifluoromethyl)thio)benzo[d]thiazole 12 (118 mg, 0.5 mmol, 1 equiv.) and pentanol as co-solvent (1.25 mL, 33%) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 24.

This result shows that the cleavage of 12 is achieved with a high yield in presence of an alcohol nucleophile.

### Example 18: Selective Cleavage of C—SCF₃ Bond in the Presence of an Ester as a Nucleophile

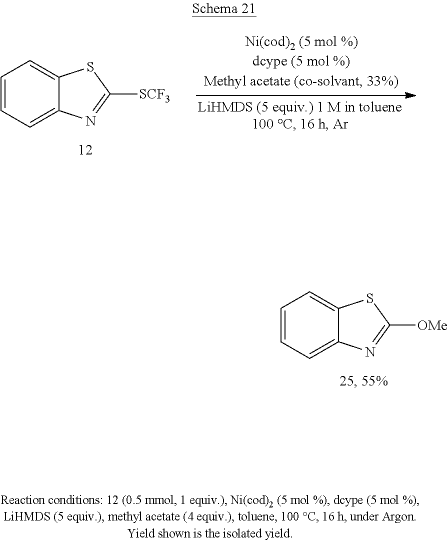

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS in toluene (2.5 mL, 1M, 418 mg, 2.5 mmol, 5 equiv.), 2-((trifluoromethyl)thio)benzo[d]thiazole 12 (118 mg, 0.5 mmol, 1 equiv.), and methyl acetate (1.25 mL, 33%) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 25.

### Example 19: Selective Cleavage of C—SCF₃ Bond in the Presence of an Ester as Nucleophile

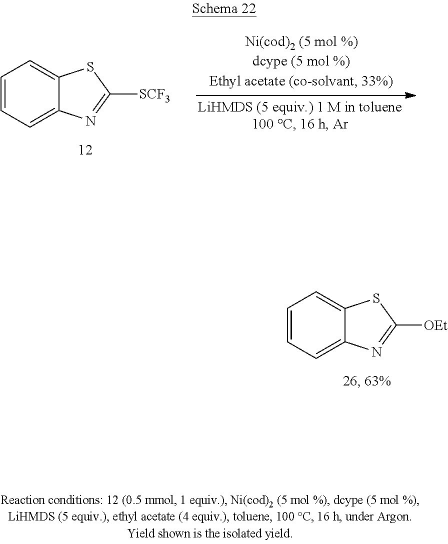

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS in toluene (2.5 mL, 1M, 418 mg, 2.5 mmol, 5 equiv.), 2-((trifluoromethyl)thio)benzo[d]thiazole 12 (118 mg, 0.5 mmol, 1 equiv.), and ethyl acetate (1.25 mL, 33%) under argon. The resulting solution was stirred at 100 °C for 16 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 26.

Examples 17, 18 and 19 illustrated the possibility of forming a C—O bond from the cleavage of 12 using different nucleophiles.

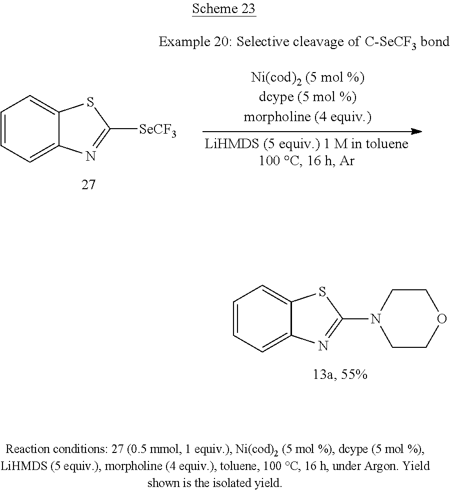

An oven-dried 10 mL tube equipped with a stirring bar was charged with Ni(cod)₂ (7 mg, 0.025 mmol, 5 mol %), dcype (11 mg, 0.025 mmol, 5 mol %), LiHMDS in toluene (2.5 mL, 1M, 418 mg, 2.5 mmol, 5 equiv.), 27 (142 mg, 0.5 mmol, 1 equiv.) and morpholine (2 mmol, 4 equiv.) under argon. The resulting solution was stirred at 100 °C for 16 h. The mixture was allowed to cooled down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 13a.

This result shows that the selective cleavage of the C—SeCF₃ bond is achieved in the presence of an amine as a nucleophile.

### Example 21: Selective Cleavage of C—SCF₃ Bond on an Aryl Group

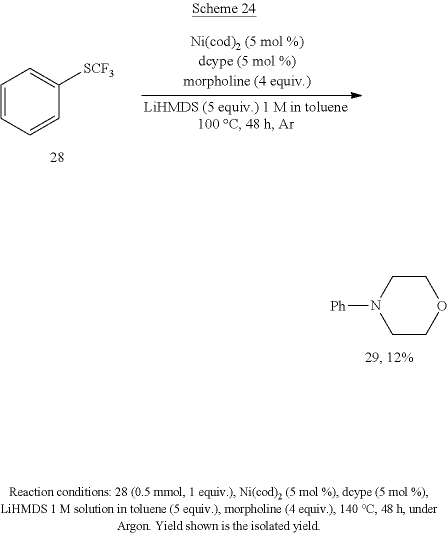

An oven-dried 10 mL tube equipped with a stirring bar was charged with Ni(cod)₂ (7 mg, 0.025 mmol, 5 mol %), dcype (11 mg, 0.025 mmol, 5 mol LiHMDS in toluene (2.5 mL, 1M, 418 mg, 2.5 mmol, 5 equiv), 28 (0.5 mmol, 1 equiv.) and morpholine (2 mmol, 4 equiv.) under argon. The resulting solution was stirred at 140 °C for 48 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 29.

### Example 22: Selective Cleavage of C—SCF₃ Bond on a Vinyl Group

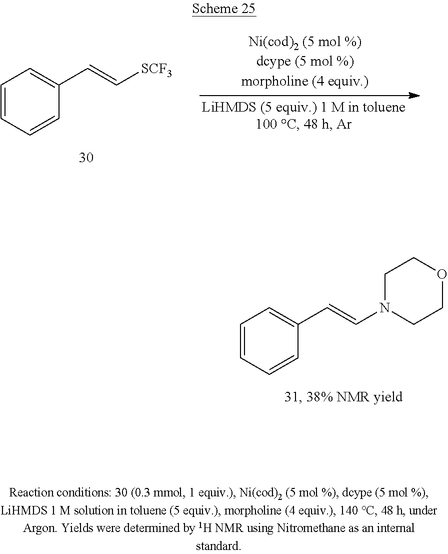

An oven-dried 10 mL tube equipped with a stirring bar was charged with Ni(cod)₂ (4 mg, 0.015 mmol, 5 mol %), dcype (6 mg, 0.015 mmol, 5 mol %), LiHMDS in toluene (1.5 mL, 1M, 251 mg, 1.5 mmol, 5 equiv.), 30 (0.3 mmol, 1 equiv.) and morpholine (1.2 mmol, 4 equiv.) under argon. The resulting solution was stirred at 140 °C for 48 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The yield of 31 was determined by ¹H NMR using Nitromethane as an internal standard.

What is claimed is:

1. A process for the preparation of a compound having the formula (I):
A-R
by reacting a compound having the formula (II):
A-X
in the presence of a metallic complex comprising nickel, palladium or rhodium(I), a ligand, a nucleophile carrying one group R, and a solvent,
R being a hydrocarbon group, optionally including at least one heteroatom and/or at least one atom other than C or H,
wherein:
A is selected from the group consisting of:
(C₆-C₁₀)aryl groups,
said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
Rₐ and R_{b} being independently from each other H or a (C₁-C₆)alkyl group;
R_{c} being a (C₁-C₆)alkyl group;
heteroaryl groups comprising from 5 to 10 atoms and including at least one heteroatom selected from O, N, and S,
said heteroaryl groups being optionally substituted with at least one substituent preferably selected from: halogen, (C₁-C₆)alkyl, (C₁-C₆)alkoxy, —CN, —CF₃, —OCF₃, —NR^{a}R^{b}, —Si(R_{c})₃, —BR^{a}R^{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
a vinyl compound having the following formula (III): 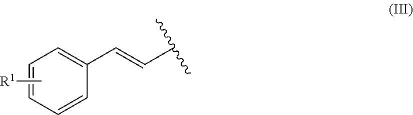
R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
X is selected from the group consisting of: YCF₃, YOCF₃, YO₂CF₃, YCF₂SO₂Ph, YOCF₂SO₂Ph, YO₂CF₂SO₂Ph, Y(O)ₙCF₂COOR′, Y(O)ₙCF₂CONR′₂, Y(O)ₙCF₂CH₂OH, Y(O)ₙCN, Y(O)ₙCHF₂, Y(O)ₙCF₂H, Y(O)ₙCF₂CₙF₂ₙ₊₁, Y(O)ₙCF₂COR′, Y being S or Se,
n being 1 or 2, and R′ being (C₁-C₆)alkyl.

2. The process of claim 1, wherein the metallic complex comprises nickel.

3. The process of claim 1, wherein the ligand is bound to the metal in the initial state of the metallic complex and/or the ligand is added to a reaction medium in addition to the metallic complex.

4. The process of claim 1, wherein the nucleophile is selected from the group consisting of: alkyl amines, cycloalkyl amines, aryl amines, alkyl thiols, thiophenol compounds, aryl selenol compounds, phosphine oxides, Grignard reagents, alcohols, esters and phosphorus derivatives.

5. The process of claim 1, wherein the nucleophile is a RH group, wherein R is selected from the group consisting of: (C₁-C₆)alkyl, heterocycloalkyl, —S-cyclo(C₃-C₁₀)alkyl, —O—(C₁-C₁₂)alkyl, an optionally substituted heteroaryl, a cyclo(C₃-C₁₀)alkylamino group, a —S—(C₁-C₆)alkyl where the alkyl is optionally substituted, —NR¹R², —P(═O)—R¹R², —X¹—Ar¹, —X¹—(C₁-C₁₂)alkyl-Ar¹, and —X¹-Het¹, —X¹—(C₁-C₁₂)alkyl-Het¹, X¹ being S, Se or NH, Ar¹ being an optionally substituted (C₆-C₁₀)aryl group, Het¹ being an optionally substituted heteroaryl group and R¹ and R² being, independently from each other, a (C₁-C₆)alkyl group or a (C₆-C₁₀)aryl group.

6. The process of claim 1, wherein the nucleophile is a RH group, wherein R is selected from the group consisting of: (C₁-C₆)alkyl, heterocycloalkyl, —S-cyclo(C₃-C₁₀)alkyl, —P(═O)—R¹R², and —X¹—Ar¹, X¹ being S, Se or NH, and Ar¹ being an optionally substituted (C₆-C₁₀)aryl group, and R¹ and R² being, independently from each other, a (C₁-C₆)alkyl group or a (C₆-C₁₀)aryl group.

7. The process of claim 1, wherein A is selected from the heteroaryl groups comprising from 5 to 10 atoms and including at least one heteroatom selected from O, N, and S, said heteroaryl groups being optionally substituted with at least one substituent selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R, being as defined in claim 1.

8. The process of claim 1, further comprising the use of a hydride compound.

9. The process of claim 1, wherein the ligand is selected from the group consisting of: 1,5-cyclooctadiene (COD), 1,2-Bis(dicyclohexylphosphino)ethane (dcype), 1,1′-Bis(diphenylphosphino)ferrocene (dppf), 1,1′-Bis(di-tert-butylphosphino)ferrocene (DTBPF), 4,5-Bis(diphenylphosphino)-9,9-dimethylxanthene (XantPhos), triphenylarsine (AsPh₃) and triphenylphosphane (PPh₃).

10. The process of claim 1, wherein the solvent is toluene.

11. The process of claim 1, wherein the reaction is carried out at a temperature from 90 °C to 140 °C.

12. The process of claim 1, wherein the metallic complex is a Ni(0) complex.

13. The process of claim 1, for the preparation of a compound having the formula (I-1): 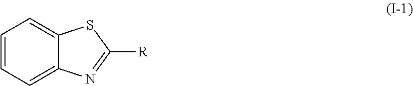
R being as defined above in claim 1,
from a compound having the formula (II-1): 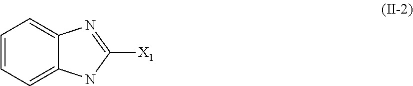
X₁ being selected from the group consisting of: SCF₃, SOCF₃, SO₂CF₃, SCF₂SO₂Ph, SOCF₂SO₂Ph, SO₂CF₂SO₂Ph, and SeCF₃.

14. The process of claim 8, wherein the hydride compound is LiHMDS.

15. The process of claim 12, wherein the metallic complex is bis(1,5-cyclooctadiene)nickel.
